(19) United States
(12) Patent Application Publication
(10) Pub. No.: US 2010/0247646 A1
(43) Pub. Date: Sep. 30, 2010

(54) EXTENDED RELEASE TABLETS OF NISOLDIPINE

(75) Inventors: Ramprasad PRASAD, Pune (IN); Anuj Kumar FANDA, Ghaziabad (IN); Rajan Kumar VERMA, New Delhi (IN); Romi Barat SINGH, Varanasi (IN); Kumaravel VIVEK, Chennai (IN)

Correspondence Address: Ranbaxy Inc., Intellectual Property Department, 600 College Road East, PRINCETON, NJ, 08540, US

(73) Assignee: RANBAXY LABORATORIES LIMITED, New Delhi (IN)

(21) Appl. No.: 12/732,941

(22) Filed: Mar. 26, 2010

(30) Foreign Application Priority Data
Mar. 26, 2009 (IN) 598/DEL/2009

(51) Int. Cl. A61K 9/22; A61K 31/455; A61P 9/12

(52) U.S. Cl. 424/468; 514/356

(57) ABSTRACT

The present invention relates to extended release tablets comprising a core which comprises of nisoldipine, a rate-controlling hydrophilic polymer and optionally, an enteric agent. Such tablets may also be coated with a release rate-controlling coating comprising of a hydrophobic polymer or enteric agent or combinations thereof, in particular ethylcellulose, and Eudragits.

### FIELD OF THE INVENTION

The present invention relates to extended release tablets comprising nisoldipine and processes for the preparation thereof.

### BACKGROUND OF THE INVENTION

Nisoldipine is a practically water-insoluble calcium channel blocker of the dihydropyridine class, chemically known as 3,5-pyridinedicarboxylic acid, 1,4-dihydro-2,6-dimethyl-4-(2-nitrophenyl)-, methyl 2-methyl-propyl ester. It is commercialized in the United States of America, under the proprietary name Sular® from Sciele Pharma, Inc. Sular® tablets are indicated for the treatment of hypertension. It may be used alone or in combination with other antihypertensive agents. Therapy usually should be initiated with 17 mg orally once daily, and then increased by 8.5 mg per week or longer intervals, to attain adequate control of blood pressure. The usual maintenance dosage is 17 mg to 34 mg once daily. U.S. Pat. Nos. 4,988,717 and 4,154,839 disclose nisoldipine and processes for the preparation thereof.

Since February 1995, Sular® has been available 10, 20, 30, and 40 mg strengths. This formulation consisted of an external coat and an internal core; wherein both the coat and core contained nisoldipine. The coat acted as a slow release formulation and the core as a fast release formulation. Recently, Sciele Pharma has replaced the 10, 20, 30 and 40 mg strengths with a bioequivalent new formulation of Sular® extended release tablets in strengths of 8.5, 17, 25.5, or 34 mg. These tablets comprise of three layers: a top barrier layer, a middle layer containing nisoldipine, and a bottom barrier layer. The top and bottom erodible barrier layers and the hydrogel middle layer provide for the controlled release of the drug.

U.S. Pat. No. 4,892,741 describes a press coated tablet comprising a core which contains a dihydropyridine in rapid-release form, and a coat around the core; the coat containing a dihydropyridine in slow-release form. However, studies have been reported showing there are slightly more adverse effects associated with such systems when compared to other drug delivery systems. U.S. Pat. No. 4,966,772 also describes a tablet having long-lasting action and a core coat construction, the core containing at least one sparingly soluble dihydropyridine active compound in slow-release form. The coat surrounding the core contains no active compound, dissolves slowly and contains a hydrophilic gel forming polymer. The tablet may be further coated with a rapid-release initial dose of the active compound mixed with a polymer, the core containing at least 50% of the amount of active compound in delayed release form. The manufacturing of such formulations involve multiple functional coating steps, which are not only cumbersome from an industrial point of view, but also may lead to variable release profiles of the active ingredient.

The prior art also exhaustively discloses multilayer tablet formulations which comprise a top barrier layer, a middle layer containing active therapeutic agent, and a bottom barrier layer. U.S. Pat. No. 5,422,123 describes such formulations which relate to a system for the controlled-rate release of active substance containing a deposit-core of swellable and gellable polymer(s), and a support-platform applied to said deposit-core, which is elastic in nature so as to partially cover the surface of the deposit-core, allows for changes due to hydration of the deposit-core, and is slowly soluble and/or slowly gellable in aqueous fluids.

U.S. Patent Publication No. 20080063711 also discloses a controlled release solid oral dosage formulation comprising a core or central layer comprising a calcium channel blocker, like nisoldipine; and one or more barrier layers comprising one or more swellable, erodible, or gellable polymers, delaying release to a region within the gastrointestinal tract providing greater uptake of the calcium channel blocker when compared to a formulation comprising only an enteric coating on the core or central layer. However, the manufacturing of such multilayered formulations require the use of complex multilayer tabletting machinery, which is a fairly expensive and slow process. Further, as in case of multilayer tablet preparations, the layers are adhered to each other by compression and during the shelf life they may be separated relatively easily by improper handling.

Therefore, there still exists a need to formulate an extended release formulation of nisoldipine which will provide for sustained release of the drug with diminished or no adverse effects and can be formulated conveniently, using simple, fast and cost-effective processes and which does not involve multiple coating steps or the formation of multiple layers.

### SUMMARY OF THE INVENTION

In one general aspect the present invention provides for an extended release tablet, which includes:
a) a core, including nisoldipine, a hydrophilic polymer and optionally, an enteric agent; and
b) a release rate-controlling coating including a hydrophobic polymer, an enteric agent or a combination thereof.

Embodiments of the present invention may include one or more of the following features. For example, the coating is substantially free of nisoldipine or the coating may also include a channel forming agent.

The hydrophilic polymer may be methylcellulose, hydroxymethyl cellulose, hydroxyethyl cellulose, hydroxypropyl cellulose, hydroxybutylcellulose, hydroxyethyl methylcellulose, hydroxypropyl methylcellulose, carboxymethylcellulose; sodium carboxymethylcellulose, carboxymethyl ethylcellulose, polyacrylic acids and the salts thereof, polymethacrylic acids and the salts thereof, methacrylate copolymers, polyvinylalcohol, polyvinylpyrrolidone, copolymers of polyvinylpyrrolidone with vinyl acetate, combinations of polyvinylalcohol and polyvinylpyrrolidone; polyalkylene oxides such as polyethylene oxide and polypropylene oxide and copolymers of ethylene oxide and propylene oxide, and combinations thereof. For example, the hydrophilic polymer may be hydroxypropyl methylcellulose.

The enteric agent may be cellulose acetate phthalate, hydroxypropyl cellulose phthalate, hydroxypropyl methylcellulose phthalate, hydroxypropyl methylcellulose acetate succinate, polyvinyl acetate phthalate, acrylic acid polymers and copolymers, and methacrylic polymers and copolymers, alginates, methacrylate-methacrylic acid co-polymers, polyvinyl acetate phthalate, styrol maleic acid copolymers, cellulose acetate phthalate; cellulose acetate trimelliate, hydroxypropyl methylcellulose acetate succinate; polymers and copolymers of methacrylic acid and methacrylates and combinations thereof.

The hydrophobic polymer may be ethyl cellulose, cellulose acetate, acrylic acid polymers and copolymers, methacrylates, polyethylenes, waxes, shellac, hydrogenated vegetable oils, high molecular weight polyvinyl alcohols, waxes and combinations thereof. For example, the hydrophobic polymer may be ethyl cellulose.

The channel forming agent may be water-soluble compounds and hydrophilic polymers, alkali metal salts, alkaline earth metals, polyglycols; ethyl vinyl alcohols; glycerin; pentaerythritol; polyvinyl alcohols; vinylpyrrolidone; N-methylpyrrolidone; saccharides; hydrolyzed starch; pregelatinized starch; carbohydrates; and sugar alcohols; and combinations thereof. For example, the channel forming agent may be hydroxypropyl methylcellulose.

### DETAILED DESCRIPTION OF THE INVENTION

The term “extended release tablet” as referred to herein is defined to mean oral pharmaceutical tablet formulations, which when administered, releases the active medicament at a relatively constant rate and provides plasma concentrations of the active medicament that remain substantially invariant over time within the therapeutic range of the active medicament over a 24-hour period. This term also encompasses “prolonged release”, “controlled release”, “modified release”, “delayed release” and “sustained release” tablets. The tablets according to the present invention deliver a therapeutically effective amount of nisoldipine to a patient for 24 hours following a once-daily administration. The term “therapeutically effective amount” intends to describe an amount of the active agent which stops or reduces the progress of the condition to be treated or which otherwise completely or partly cures or acts palliatively on the condition. Nisoldipine or a pharmaceutically acceptable salt or derivative, analogue or polymorph thereof may be present in an amount of about 1 mg to about 200 mg. The recommended doses of Sular° may also be considered as a therapeutically effective dose. In particular embodiments, nisoldipine is present in amounts of 8 mg, 17.5 mg, 25.5 mg, and 34 mg.

The extended release tablet as described herein may comprise of a core comprising nisoldipine, a hydrophilic polymer and optionally, an enteric agent. The said core may be in the form of tablets, minitablets, granules, pellets, beads or pills.

The recital of the term “uncoated” renders that the said extended release tablet does not allow for the inclusion of a functional coat, barrier, layer or the like, in a way so as to control, sustain, delay, extend, prolong or modify the release of nisoldipine from the core tablet. The said uncoated tablet however, may optionally be coated using conventional non-functional coating compositions, which are available under the trade name of Opadry®, Opaspray®, from Colorcon Limited, UK.

A “hydrophilic polymer” as disclosed herein includes without limitation those polymers and agents that swell and/or gel in the aqueous media. Hydrophilic polymers suitable for use in the core include alkylcelluloses, such as methylcellulose; hydroxyalkylcelluloses, for example, hydroxymethyl cellulose, hydroxyethyl cellulose, hydroxypropyl cellulose and hydroxybutylcellulose; hydroxyalkyl alkylcelluloses, such as hydroxyethyl methylcellulose and hydroxypropyl methylcellulose; carboxyalkylcelluloses, such as carboxymethylcellulose; alkali metal salts of carboxyalkylcelluloses, such as sodium carboxymethylcellulose; carboxyalkyl alkylcelluloses, such as carboxymethyl ethylcellulose; carboxyalkylcellulose esters; other natural, semi-synthetic, or synthetic polysaccharides and gums, such as alginic acid, alkali metal and ammonium salts thereof, carrageenans, galactomannans, tragacanth, agar-agar, gum arabic, guar gum, xanthan gum, starches, pectins, such as sodium carboxymethyl amylopectin, chitin derivates, such as chitosan, polyfructans, inulin; polyacrylic acids and the salts thereof; polymethacrylic acids and the salts thereof, methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides, such as polyethylene oxide and polypropylene oxide and copolymers of ethylene oxide and propylene oxide. In a particular embodiment, the hydrophilic polymer(s) are the alkyl celluloses, like hydroxypropyl methylcellulose. Suitable types are sold under the trade name of Methocel® by The Dow Chemical Company such as Methocel® K4MCR, and Methocel® K100MCR.

The amount of hydrophilic polymer present may vary from about 1% (w/w) to about 90% (w/w), particularly about 1% (w/w) to about 75% (w/w), and more particularly about 1% (w/w) to about 50% (w/w) of the core. The amount of hydrophilic polymer present in the core may vary from about 5% to about 80% by weight of the total weight of the tablet, particularly from about 5% (w/w) to about 70% (w/w), more particularly from about 5% (w/w) to about 50% (w/w) by weight of the total weight of the tablet.

Optionally, the core may comprise of an enteric agent that in combination with the hydrophilic polymer may further help in providing the desired drug release profile from the extended release tablet. The enteric agent may include cellulose polymers, such as cellulose acetate phthalate, hydroxypropyl cellulose phthalate, hydroxypropyl methylcellulose phthalate and hydroxypropyl methylcellulose acetate succinate; polyvinyl acetate phthalate, acrylic acid polymers and copolymers, and methacrylic polymers and copolymers that are commercially available under the trade name Eudragit® (Evonik-Degussa), such as copolymers of ethyl acrylate, methyl methacrylate with a low content of a methacrylic acid ester with quaternary ammonium groups (trimethyl ammonioethyl methacrylate chloride) (Eudragit® RS100 and Eudragit® RL100) and poly(ethylacrylate-methylmethacrylate) (Eudragit® NE); alginates, alkali-soluble acrylic resins, hydroxypropyl methylcellulose phthalate, methacrylate-methacrylic acid co-polymers, polyvinyl acetate phthalate, styrol maleic acid copolymers, and the like, and combinations thereof. The amount of the enteric agent that may be present in the core is from about 0.1% (w/w) to about 25% (w/w) by total weight of the tablet.

The core of the extended release tablet of the invention may further include one or more of other pharmaceutically acceptable excipient(s). The term “pharmaceutically acceptable excipient(s)” as recited herein includes conventional pharmaceutical additives known in the art, such as diluent(s), binder(s), solubilizer(s), lubricants(s), granulating solvent(s), glidants(s) or combinations thereof.

Suitable diluents that may be used include saccharides, for example, lactose, dextrose, sucrose, fructose, maltose; sugars, for example, mannitol, erythritol, sorbitol, xylitol and lactitol; cellulose derivatives, for example, powdered cellulose, microcrystalline cellulose; dicalcium phosphate, tribasic calcium phosphate, calcium sulphate, calcium carbonate, kaolin and the like.

Suitable binders that may be used include, starch derivatives, for example, corn starch and pregelatinized starch; cellulose ethers, such as carboxymethyl cellulose, methylcellulose, hydroxypropyl cellulose, hydroxypropyl methylcellulose; carboxy vinyl polymers, for example, carbomers; acrylates, such as Eudragits; polyvinylpyrrolidone, polyvinylpyrrolidone/vinyl acetate copolymer; xanthan gum, guar gum and other such materials routinely used in the art of solid dosage form manufacturing.

Suitable solubilizer(s) that may be used include sodium, potassium, ammonium of long chain alkyl sulfonates and alkyl aryl sulfonates, such as sodium dodecylbenzene sulphonate; dialkyl sodium sulphosuccinates; dialkyl sodium sulfosuccinates; alkyl sulphates, such as sodium lauryl sulphate; PEG-150 laurate; PEG-400 monolaurate; polyoxyethylene monolaurate; polysorbates; polyethoxylated castor oil (e.g. Cremophor®), benzalkonium chloride; benzethonium chloride; cetrimonium bromide, stearyl dimethylbenzyl ammonium chloride, ethylene glycol monostearate, propylene glycol myristate, glyceryl monostearate, glyceryl stearate, polyglyceryl-4-oleate, sorbitan acylate, sucrose acylate, polyoxyethylene octylphenylether, PEG-1000 cetyl ether, polyoxyethylene tridecyl ether, polypropylene glycol butyl ether, Poloxamer®, stearoyl monoisopropanolamide, polyoxyethylene hydrogenated tallow amide, sodium N-dodecyl-β-alanine, sodium N-lauryl-β-iminodipropionate, myristoamphoacetate, lauryl betaine and lauryl sulfobetaine.

Suitable lubricants include magnesium stearate, calcium stearate, zinc stearate, sodium stearyl fumarate, powdered stearic acid, magnesium oleate, calcium palmitate, potassium laureate, sodium suberate, vegetable oil, mineral oil and the like. Suitable glidants may include talc, colloidal silicon dioxide, corn starch and the like.

Suitable granulating solvents that may be used include water, ethanol, methanol, isopropyl alcohol, methylene chloride, acetone, and combinations or equivalents thereof.

The “release rate-controlling coating” of the extended release tablet as described herein, surrounds the core entirely and completely. The coating comprises one or more hydrophobic polymer(s), one or more enteric agent(s), or combinations thereof. The one or more hydrophobic polymers which may be used in the coating include polymers, such as ethyl cellulose, cellulose acetate, acrylic acid polymers and copolymers, methacrylates, polyethylenes, waxes, shellac, hydrogenated vegetable oils, high molecular weight polyvinyl alcohols and waxes, such as fatty acids, glycerides, glyceryl esters, for example, glyceryl behenate, and combinations thereof. In one embodiment ethylcellulose is used in the coating.

The one or more enteric agent(s) that may be used in the coating may be identical or different to the enteric agent that has been used in the core (if any). Such enteric agent may include one or more of cellulose acetate phthalate; cellulose acetate trimelliate; hydroxypropyl methylcellulose phthalate; hydroxypropyl methylcellulose acetate succinate; polyvinyl acetate phthalate; acrylic acid polymers and copolymers; polymers and copolymers of methacrylic acid and methacrylates (e.g. those available under the trade name Eudragit® from Evonik-Degussa). In one embodiment, methacrylate based polymers (e.g. Eudragit® L100-55 and Eudragit® L30D-55) are used.

The amount of the one or more hydrophobic polymer and/or enteric agent in the tablet may be in an amount of about 1% (w/w) to about 25% (w/w) of the total weight of the extended release tablet. The hydrophobic polymer and/or enteric agent may be present in an amount of about 1% (w/w) to about 95% (w/w), particularly about 1% (w/w) to about 80% (w/w) of the coating composition.

The recital of “the said coating is substantially free of nisoldipine” renders that the said coating does not contain nisoldipine, or, if any nisoldipine is present it is not present in an amount to provide an initial therapeutic, pharmaceutical or burst effect.

The coating composition as described herein may further comprise of one or more additives including water soluble agents, channel forming agents, plasticizers, coloring agents, anti-tacking agents, lubricants/glidants, solvents and other conventionally used coating additives.

Suitable water soluble agent(s) that may be used include cellulosic polymers, such as hydroxypropyl methylcellulose; polyvinylpyrrolidone; vinyl acetate copolymers; starch and starch based polymers; polysaccharides; or a mixture thereof.

Suitable channel forming agents that may be used include water-soluble compounds and hydrophilic polymers, and may include alkali metal salts, e.g. sodium chloride, sodium bromide and the like; alkaline earth metals, e.g. calcium phosphate, calcium nitrate and the like; transition metal salts, e.g. ferric chloride, ferrous sulfate and the like; polyglycols; ethyl vinyl alcohols; glycerin; pentaerythritol; polyvinyl alcohols; vinylpyrrolidone; N-methyl pyrrolidone; saccharides; hydrolyzed starch; pregelatinized starch; carbohydrates, for e.g. glyceraldehydes, erythrose, ribose, arabinose, xylose, glucose, mannose, galactose, maltose, lactose, sucrose and the like; and sugar alcohols, e.g. mannitol and the like. Hydrophilic polymer(s) that may be used as a channel forming agent may include hydroxypropyl cellulose, hydroxypropyl methylcellulose, polyvinylpyrrolidone and the like.

Suitable plasticizers include dibutyl sebacate, polyethylene glycol, triethyl citrate, triacetin, acetylated triacetin, tributyl citrate, glycerol tributyrate, natural, semi-synthetic and synthetic glycerides, monoglyceride, acetylated monoglycerides, fractionated coconut oil, rape oil, olive oil, sesame oil, castor oil, hydrogenated castor oil, acetyltributylcitrate, acetyltriethylcitrate, glycerin sorbitol, diethyl oxalate, diethyl phthalate, diethyl malate, diethyl fumarate, dibutyl succinate, diethyl malonate, dioctyl phthalate, and the like.

Suitable anti-tacking agents include adipic acid, magnesium stearate, calcium stearate, zinc stearate, hydrogenated vegetable oils, Sterotex®, glyceryl monostearate, talc, silica, sodium benzoate, sodium lauryl sulfate, magnesium lauryl sulfate, and the like.

The coating composition may further include one or more solvents. Suitable solvents for the coating composition include various solvents such as isopropyl alcohol (IPA), water, methylene chloride and mixtures thereof. Various combinations of solvents may also be used, such as isopropyl alcohol and water, isopropyl alcohol and methylene chloride and the like.

The extended release tablet may further be coated with a non-functional coating. The non-functional coating may additionally include one or more pharmaceutically acceptable colourants or opacifiers, including water soluble dyes, aluminium lakes of water soluble dyes and inorganic pigments such as titanium dioxide and iron oxide. It may also contain one or more plasticizing agents conventionally used in polymeric film coatings, for example, polyethylene glycol, propylene glycol, dibutyl sebacate, mineral oil, sesame oil, diethyl phthalate and triacetin. Proprietary non-functional coating materials, such as Opaspray® and Opadry®, obtainable from Colorcon Limited, UK, may also be used.

The core of extended release tablet or the uncoated extended release tablet as described herein may be formulated following any conventional techniques known in the art, namely dry granulation, aqueous or non-aqueous wet granulation, melt granulation, direct compression, roller compactation, pelletization, melting, intimate and non-intimate blending, kneading, and the like.

The core comprising nisoldipine as described herein above may be coated with the coating composition(s) using various techniques, including conventional coating techniques, such as perforated pan, fluidized bed technique, compression coating or spraying.

In one embodiment, the extended release tablet includes:
a) a core comprising nisoldipine and a hydrophilic polymer; and
b) a release rate-controlling coating comprising of an enteric agent, a plasticizer, and an anti-tacking agent.

In another embodiment, the extended release tablet may include:
a) a core comprising nisoldipine and a hydrophilic polymer; and
b) a release rate-controlling coating comprising of a hydrophobic polymer and a channel forming agent.

In yet another embodiment, the uncoated extended release tablet comprises of nisoldipine and a hydrophilic polymer.

In one aspect of the above embodiments, the tablet core may further include one or more enteric agents. The hydrophilic polymer may be hydroxypropyl methylcellulose, hydroxypropyl cellulose or a combination thereof. The enteric agent in the coating may be a copolymer of methacrylic acid and ethyl acrylate. The hydrophobic polymer may be ethylcellulose and the channel forming agent may be hydroxypropyl methylcellulose.

From the above it is apparent that various modifications and combinations of the formulations detailed in the text may be made without departing from the spirit and scope of the invention. The invention as described herein may be illustrated by the following examples but is not to be construed to be limited by them.

### Examples 1-2

### Uncoated Tablets

Quantity per tablet (mg)

| Ingredients | Example 1 | Example 2 |
|---|---|---|
| Nisoldipine | 17.00 | 17.00 |
| Lactose monohydrate | 114.25 | 124.25 |
| Hypromellose | 40.00 | 40.00 |
| K4MCR | | |
| Hypromellose E 50 | — | 40.00 |
| LV | | |
| Hypromellose K 100 | 50.00 | — |
| LV | | |
| Sodium lauryl sulphate | 15.00 | 15.00 |
| Povidone | 10.00 | 10.00 |
| Magnesium stearate | 2.50 | 2.50 |
| Colloidal silicon | 1.25 | 1.25 |
| dioxide | | |
| Total Weight | 250.00 | 250.00 |

### Procedure:

1. Nisoldipine, hypromellose (in different grades as specified in Examples 1 and 2), lactose monohydrate, povidone, and sodium lauryl sulphate were sifted through BSS# 36 mesh.
2. The blend of step 1 was screened, and lubricated with colloidal silicon dioxide and magnesium stearate in a low shear blender.
3. The lubricated mass of step 3 was compressed using appropriate tooling.

### Examples 3-5

### Tablets with Hydrophobic Polymer Coating

### Core Tablets:

| | Quantity per tablet (mg) | | |
|---|---|---|---|
| Ingredients | Example 3 | Example 4 | Example 5 |
| Nisoldipine | 34.00 | 17.00 | 17.00 |
| Lactose monohydrate | 308.50 | 154.25 | 149.25 |
| Hypromellose K4MCR | 100.00 | 50.00 | 50.00 |
| Povidone K 30 | 20.00 | 10.00 | 10.00 |
| Sodium lauryl sulphate | 30.00 | 15.00 | 20.00 |
| Magnesium stearate | 5.00 | 2.50 | 2.50 |
| Colloidal silicon dioxide | 2.50 | 1.25 | 1.25 |
| Purified Water | q.s. | q.s. | q.s. |
| Total Weight | 500.00 | 250.00 | 250.00 |

### Procedure:

1. Nisoldipine, hypromellose, lactose monohydrate and sodium lauryl sulphate were sifted through BSS# 36 mesh.
2. The blend of step 1 was dry mixed in a rapid mixer granulator and granulated using an 18% w/w aqueous binder solution of povidone.
3. The wet mass of step 2 was dried in a fluidized bed dryer.
4. The dried granules of step 3 were milled through a Quadro® co-mill fitted with 40G screen.
5. The milled granules of step 4 were lubricated with colloidal silicon dioxide and magnesium stearate in a low shear blender for 5 minutes.
6. The lubricated granules of step 5 were compressed using appropriate tooling into core tablets.

### Rate Controlling Coating Composition (Hydrophobic Polymer):

Quantity per
tablet (mg)

| Ingredients | A | B |
|---|---|---|
| Ethyl cellulose 7 cps | 30.00 | 20.00 |
| Hypromellose E5 | 20.00 | 30.00 |
| Isopropyl alcohol | q.s. | q.s. |
| Purified Water | q.s. | q.s. |

### Procedure:

The core tablets obtained in Examples 3, 4 and 5 were coated using an isopropyl alcohol:water mixture (70:30% w/w) based ethyl cellulose coating composition comprising of ethyl cellulose 7 cps and hypromellose E5. Two batches of the core tablets were prepared according to Example 3; of which one batch was coated using composition A and the other one was coated using composition B to obtain final extended release tablets 3A and 3B respectively. The core tablets in Examples 4 and 5 were coated using coating composition B to obtain final extended release tablets 4B and 5B respectively.

### Example 6

### Tablets with Enteric Agent Coating

### Core Tablets:

| | Quantity per tablet |
|---|---|
| Ingredients | (mg) |
| Nisoldipine | 34.00 |
| Lactose monohydrate | 233.50 |
| Hypromellose K4MCR | 175.00 |
| Povidone K 30 | 20.00 |
| Sodium lauryl sulphate | 30.00 |
| Magnesium stearate | 5.00 |
| Colloidal silicon dioxide | 2.50 |
| Purified Water | q.s. |
| Total Weight | 500.00 |

### Procedure:

1. Nisoldipine, hypromellose, lactose monohydrate and sodium lauryl sulphate were sifted through BSS# 36 mesh.
2. The blend of step 1 was dry mixed in a rapid mixer granulator and granulated using an 18% w/w aqueous binder solution of povidone.
3. The wet mass of step 2 was dried in a fluidized bed dryer.
4. The dried granules of step 3 were milled through Quadro® co-mill fitted with 40G screen.
5. The milled granules of step 4 were lubricated with colloidal silicon dioxide and magnesium stearate in a low shear blender for 5 minutes.
6. The lubricated granules of step 5 were compressed using appropriate tooling into core tablets.

### Rate Controlling Coating Composition (Enteric Agent):

| | Quantity per tablet |
|---|---|
| Ingredients | (mg) |
| Eudragit L30D-55 ™ | 17.224 |
| Polyethylene glycol 6000 | 2.592 |
| Triethyl citrate | 1.296 |
| Colloidal Silicon dioxide | 3.888 |
| Purified Water | q.s. |

### Procedure:

The core tablets obtained in Example 6 were coated using an aqueous coating composition comprising Eudragit® L30D-55, triethyl citrate, Polyethylene glycol 6000, and colloidal silicon dioxide.

Tablets of Examples 1, 2, 3A, 3B, 4B, 5B and 6 were subjected to dissolution studies in a USP II apparatus in 900 mL of pH 6.5 phosphate buffer with 1% sodium lauryl sulphate. The temperature and agitation were set at 37° C.±0.5° C. and 100 rpm, respectively. Dissolution profiles of these tablets are provided in Table 1.

TABLE 1
In vitro release pattern of nisoldipine from extended release tablets prepared
as per composition of Examples 1, 2, 3A, 3B, 4B, 5B and 6 in USP II
apparatus in 900 mL of pH 6.5 phosphate buffer with 1% sodium lauryl
sulphate (37° C. ± 0.5° C. and 100 rpm)

| | Percent of drug released | | | | | | |
|---|---|---|---|---|---|---|---|
| Time | | | Example | Example | Example | Example | |
| (hr) | Example 1 | Example 2 | 3A | 3B | 4B | 5B | Example 6 |
| 1 | 19 | 36 | 1 | 3 | 5 | 4 | 7 |
| 2 | 33 | 52 | 4 | 13 | 23 | 13 | 16 |
| 4 | 47 | 74 | 15 | 42 | 69 | 48 | 34 |
| 6 | 59 | 97 | 30 | 75 | 102 | 89 | 48 |
| 8 | 71 | 100 | 56 | 102 | 103 | — | 63 |
| 12 | 86 | — | 106 | 104 | — | — | 86 |

1. An extended release tablet comprising:
c) a core, comprising nisoldipine, a hydrophilic polymer and optionally, an enteric agent; and
d) a release rate-controlling coating comprising a hydrophobic polymer, an enteric agent or a combination thereof.

2. The extended release tablet according to claim 1, wherein the coating is substantially free of nisoldipine.

3. The extended release tablet according to claim 1, wherein the coating further comprises a channel forming agent.

4. The extended release tablet according to claim 1, wherein the hydrophilic polymer comprises methylcellulose, hydroxymethyl cellulose, hydroxyethyl cellulose, hydroxypropyl cellulose, hydroxybutylcellulose, hydroxyethyl methylcellulose, hydroxypropyl methylcellulose, carboxymethylcellulose; sodium carboxymethylcellulose, carboxymethyl ethylcellulose, polyacrylic acids and the salts thereof, polymethacrylic acids and the salts thereof, methacrylate copolymers, polyvinylalcohol, polyvinylpyrrolidone, copolymers of polyvinylpyrrolidone with vinyl acetate, combinations of polyvinylalcohol and polyvinylpyrrolidone; polyalkylene oxides such as polyethylene oxide and polypropylene oxide and copolymers of ethylene oxide and propylene oxide, and combinations thereof.

5. The extended release tablet according to claim 4, wherein the hydrophilic polymer comprises hydroxypropyl methylcellulose.

6. The extended release tablet according to claim 1, wherein the enteric agent comprises cellulose acetate phthalate, hydroxypropyl cellulose phthalate, hydroxypropyl methylcellulose phthalate, hydroxypropyl methylcellulose acetate succinate, polyvinyl acetate phthalate, acrylic acid polymers and copolymers, and methacrylic polymers and copolymers, alginates, methacrylate-methacrylic acid co-polymers, polyvinyl acetate phthalate, styrol maleic acid copolymers, cellulose acetate phthalate; cellulose acetate trimelliate, hydroxypropyl methylcellulose acetate succinate; polymers and copolymers of methacrylic acid and methacrylates and combinations thereof.

7. The extended release tablet according to claim 1, wherein the hydrophobic polymer comprises ethyl cellulose, cellulose acetate, acrylic acid polymers and copolymers, methacrylates, polyethylenes, waxes, shellac, hydrogenated vegetable oils, high molecular weight polyvinyl alcohols, waxes and combinations thereof.

8. The extended release tablet according to claim 7, wherein the hydrophobic polymer comprises ethyl cellulose.

9. The extended release tablet according to claim 3, wherein the channel forming agent comprises water-soluble compounds and hydrophilic polymers, alkali metal salts, alkaline earth metals, polyglycols; ethyl vinyl alcohols; glycerin; pentaerythritol; polyvinyl alcohols; vinylpyrrolidone; N-methylpyrrolidone; saccharides; hydrolyzed starch; pregelatinized starch; carbohydrates; and sugar alcohols; and combinations thereof.

10. The extended release tablet according to claim 9, wherein the channel forming agent comprises hydroxypropyl methylcellulose.
